(19) United States
(12) Patent Application Publication
(10) Pub. No.: US 2004/0033282 A1
(43) Pub. Date: Feb. 19, 2004

(54) Kefir extract as anti-cancer agent

(76) Inventors: Stan Kubow, Pointe-Claire (CA); Laurie Hing Man Chan, Baie D'Urfe (CA); Chujian Chen, Jiangsu Nantong (CN); Maryam Fotouhinia, Montreal (CA)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/311,504

(22) PCT Filed: Jun. 15, 2001
(86) PCT No.: PCT/CA01/00896

(51) Int. Cl. A61K 35/68

(52) U.S. Cl. 424/780

(57) ABSTRACT

The present invention relates to an anti-cancer composition having anti-proliferative and/or inhibitory effects specifically targeted at malignant cells, which comprises a filtrated bacteria-free and/or yeast-free liquid extract of initial fermentative kefir in association with a pharmaceutically acceptable carrier. The present invention also relates to a method of inhibiting proliferation of malignant cells in patient, which comprises administering an effective amount of a filtrated bacteria-free and/or yeast-free liquid extract of initial fermentative kefir. The present invention also relates to a prophylactic composition having neutraceutical properties, which comprises a filtrated bacteria-free and/or yeast-free liquid extract of initial fermentative kefir in association with a pharmaceutically acceptable carrier.

### BACKGROUND OF THE INVENTION

(a) Field of the Invention

The invention relates to novel anti-cancer agent and uses thereof in cancer treatment.

(b) Description of Prior Art

Research on the putative health benefits of fermented milks (FM) has grown dramatically in the past 20 years. In particular, by-products of bacterial fermentation of proteins, lipids and carbohydrates present in FM have been implicated to exert health benefits beyond basic nutrition including anti-tumor action, immune system enhancement and antioxidant effects. Epidemiological studies have indicated a reduced risk of breast cancer in women who consumed FM products (Veer P et al. Cancer Res 49:4020-4023, 1989). Antimutagensis of FM has also been widely demonstrated (Abdelali H et al., Mutation Res 331:133-141, 1995). The active ingredients in the fermented milk products have not been fully characterized but several studies suggested that the antimutagenic effect of these cultured milk was due to the presence of the lactic acid bacteria (Pol-Zobel B L et al., Nutr Cancer 20:261-270, 1993). Abdelali (Abdelali H et al., Mutation Res 331:133-141, 1995) reported that the bifidobacterium sp., casein and calcium components in FM showed a dose-dependent antimutagenic activity against benzo[α]pyrene mutagenicity in the Ames test using Salmonella typhimurium TA98. In animal models, lactobacilli and bifidobacteria have been shown to inhibit the growth or cause regression of tumors, which have been transplanted or chemically induced. Shiomi et al. (Shiomi M et al., Jap J Med Sci Bio. 35:75-80, 1982) showed that polysaccharides extracted from kefir grain had antitumor activity in mice.

The FM product, kefir, enjoys a rich tradition of health claims, as consumption of kefir has been used in the former Soviet Union for the treatment of a variety of conditions including metabolic disorders, atherosclerosis, cancer, and gastrointestinal disorders (Koroleva N S. IDF Bull. 227: 35-40, 1988). In the former Soviet Union, kefir accounts for 70% of the total amount of FM consumed. Kefir distinguishes itself from the more known FM product, yogurt in that it is traditionally made only from kefir grains which contain a complex mixture of both bacteria and yeast. Hence, in kefir production the milk undergoes a dual fermentation process under the action of both lactic acid bacteria and yeasts.

While yogurt can readily be made from the lactic acid bacteria present in fresh yogurt, kefir can only be made from kefir grains and mother cultures prepared from grains. The grains contain a relatively stable and specific balance of microorganisms, which exist in a complex symbiotic relationship. The grains are formed in the process of making kefir and only from pre-existing grains. The grains include primarily lactic acid bacteria (lactobacilli, lactococci, leuconostocs) and yeast. They resemble small cauliflower florets, and each grain is 3 to 20 mm in diameter. Kefir grains are clusters of microorganisms held together by a matrix of polysaccharides. Kefiranofaciens and L. kefir produce these polysaccharides. The polysaccharides are an integral part of the grain, and without their presence, kefir grains cannot be propagated.

Encouraging results regarding an anti-tumor activity of kefir in animal studies have been reported (Shiomi M et al., Jap J Med Sci Bio. 35:75-80, 1982; Cevikbas A et al., Phytother Res 8: 78-82, 1994; Furukawa N et al., J. Jap. Soc. Food Sci. 43:450-453, 1990; Kubo M et al., Pharmacological study on kefir—a fermented milk product in Caucasus. I. On antitumor activity (1) Yakugaku Zasshi 112: 489-495, 1992). For example, oral doses of 100 or 500 mg/kg of kefir to mice with solid tumor of E-ascites carcinoma (EC) transplanted s.c. were shown to cause a significant reduction in transplanted tumor size and activate the immunosuppressive activity of the spleen (Kubo M et al., Pharmacological study on kefir—a fermented milk product in Caucasus. I. On antitumor activity (1) Yakugaku Zasshi 112: 489-495, 1992).

In particular, it is not evident from previous work: (1) whether kefir exerts an anti-proliferative effect on tumor cells and if this effect is specific to tumor cells; and (2) whether there are different anti-proliferative potencies associated with specific stages of kefir manufacture.

It would be highly desirable to be provided with a novel anti-cancer agent and uses thereof in cancer treatment.

### SUMMARY OF THE INVENTION

One aim of the present invention is to provide a novel anti-cancer agent and uses thereof in cancer treatment.

In accordance with the present invention there is provided an anti-cancer composition having anti-proliferative and/or inhibitory effects specifically targeted at malignant cells, which comprises a filtrated bacteria-free and/or yeast-free liquid extract of initial fermentative kefir in association with a pharmaceutically acceptable carrier.

The filtrated extract of the anti-cancer composition in accordance with a preferred embodiment of the present invention, is ultrafiltrated or microfiltrated.

The liquid extract of the anti-cancer composition in accordance with a preferred embodiment of the present invention, comprises a protein concentration of 300 ng/ml to 5000 ng/ml, or more preferably of about 313 ng/ml.

In accordance with the present invention there is also provided a method of inhibiting proliferation of malignant cells in patient, which comprises administering an effective amount of a filtrated bacteria-free and/or yeast-free liquid extract of initial fermentative kefir.

The malignant cells used in a method in accordance with a preferred embodiment of the present invention, are selected from the group consisting of estrogen responsive cancer, such as breast or uterine cancer, cancer induced by oncovirus, hepatic cancer, colon cancer, prostate cancer, skin cancer and lung cancer.

In accordance with the present invention there is also provided a prophylactic composition having neutraceutical properties, which comprises a filtrated bacteria-free and/or yeast-free liquid extract of initial fermentative kefir in association with a pharmaceutically acceptable carrier.

For the purpose of the present invention the following terms are defined below.

The term “kefir” is intended to mean an end-product of a kefir manufacturing process.

The term “liquid extract of initial fermentative kefir” is intended to mean an intermediate fermentation by-product of a kefir manufacturing process.

### BRIEF DESCRIPTION OF THE DRAWINGS

FIGS. 1A and 1B illustrate the effects of different extracts from different stages in the manufacture of kefir (FIG. 1A) and yogurt (FIG. 1B) on MCF-7 cells;

FIGS. 2A and 2B illustrate the effects of different extracts from different stages in the manufacture of kefir (FIG. 2A) and yogurt (FIG. 2B) on HMEC normal human mammary epithelial cells; and

FIGS. 3A and 3B illustrate a schematic representation of the kefir manufacture.

### DETAILED DESCRIPTION OF THE INVENTION

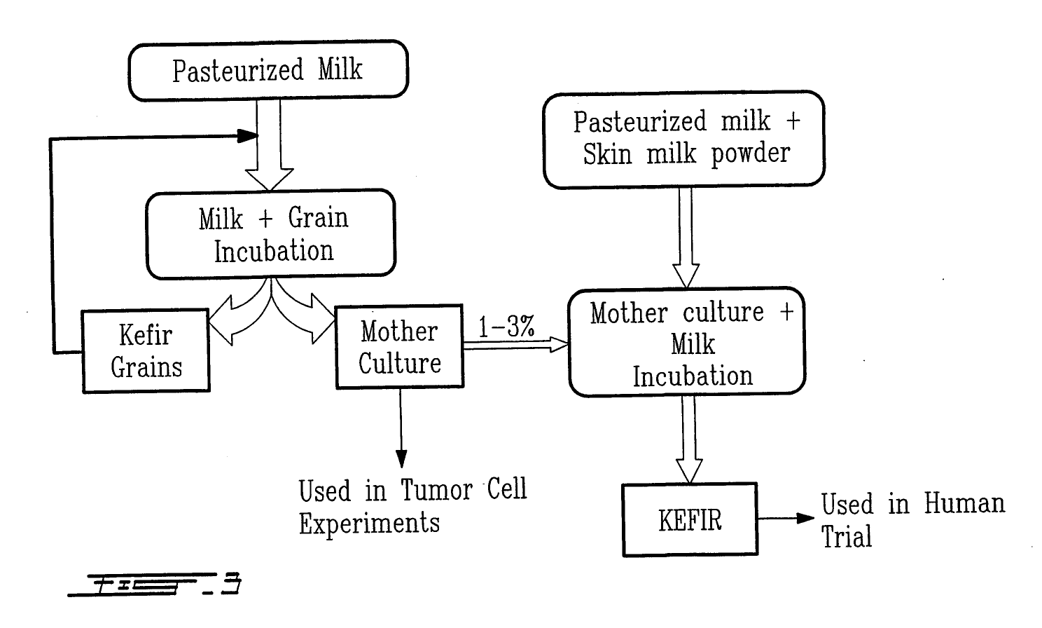

Surprisingly, and in accordance with the present invention, there is demontrated for the first time that a fraction of an early stage of kefir manufacture is associated with the most potent anti-proliferative effect on tumor cells. The kefir liquid fraction is a filtrated fraction of the mother culture as illustrated on FIG. 3. This filtrated fraction is substantially free of any bacteria and/or yeast.

Epidemiological studies have indicated that consumption of fermented milk products reduced risk of breast cancer. Effects of kefir, a traditional fermented milk product, on the growth of human mammary cancer cells have not been characterized. Both kefir and yogurt were filtered to eliminate microbes and the extracts were then incubated with normal human mammary epithelial cells and human mammary cancer (MCF-7) cells to examine their effects on cell proliferation. Both kefir and yogurt suppressed the proliferation of human MCF-7 cancer cells but the antiproliferative effects of kefir were significantly greater (p<0.01). After 8 days of culture, the kefir extract (1:640 dilution in medium) decreased MCF-7 cell numbers by 40% while yogurt extract (1:160 dilution in medium) decreased the cell numbers by only 15%. The antiproliferative effects of the two fermented milks were not accountable by lactic acid concentrations in the fermented milk extracts and were not observed in the normal human mammary epithelial cells. Milk extract had no effect on the growth of either the MCF-7 cells or the normal human mammary epithelial cells. These results indicate that kefir and yogurt extracts contain active ingredients that have antiproliferative properties on human mammary cancer cells. Unlike yogurt extracts, the kefir extracts did not suppress the growth of normal human mammary cells suggesting that the kefir extracts contain bioactive ingredients that exert a growth suppressive effect that is specific to cancer cells.

Materials and Methods:

Cell Culture:

MCF7-E3 human breast cancer estrogen-sensitive cells were provided by Dr. D. Desaulniers of Health Canada, Ottawa. Cells were routinely propagated as a monolayer culture in Dulbcco's Modified Eagle Medium (DMEM) supplemented with 10% heat-inactivated fetal bovine serum (FBS), in 75-cm² plastic dish at 37° C. in a humidified atmosphere with 5% CO₂, and passage 3-4 days a time. A normal human mammary epithelial cell line was provided by Dr. M. Stampfer of UC Livermore Labs. Cells were routinely propagated as a monolayer culture in Mammary Epithelial Growth Media (MEGM, Clonetics, San Diego) supplemented with 10% heat-inactivated fetal bovine serum (FBS), in 75-cm² plastic dish at 37 ° C. in a humidified atmosphere with 5% CO₂, and passage every week. For the experiments, both cells were harvest from the dish using 0.25% trypsin-EDTA solution.

Preparation of Extracts:

Four kefir products (K1-K4) collected at various stages of kefir production at Liberty Brand Products, Inc. (Montreal, Canada) were used in this study. The large-scale production of kefir involves a two-step fermentation process. The first step is to prepare the cultures by incubating milk with kefir grains (2-10%) (K1) and fermented for 24 hrs. The grains are then removed by filtration and the resulting mother culture (K2) is added to pasteurized milk (K3), which is further fermented for 12 hrs and this final product (K4) is packaged for the consumer market. A pasteurized milk sample (M1) and two yogurt products; mixture of yogurt bacteria, pasteurized milk and milk powder (Y1) and the final yogurt product after 12 hrs of fermentation (Y2) were included for comparison. The yeast and bacteria in the samples were removed by centrifugation and filtration. About 35 ml each of the seven samples was centrifuged (32,000×g, 60 min, 4° C.) and the supernatant was filtered through a 0.45 μm Millipore filter followed by a 0.2 μm Millipore filter (Millipore Corporation, Bedford, USA). Extracts from two separate batches of kefir and yogurt were used.

Cell Proliferation Experiments:

Cells previously harvested were seeded in 24-well plates; 10,000 cells for MCF-7 per well in DMEM supplemented with 10% FBS and 5,000 cells for HMEC per well in MEGM supplemented with 10% FBS. The cells were allowed to attach for 24 hours. After that period, old media were removed and fresh media and extracts were added to each well. To study the dose response, a serial dilution of each the extract using the culture media was made to achieve final concentrations of extracts at 1:40, 1:80, 1:160, 1:320 and 1:640 (vol/vol) or 2.5%, 1.3%, 0.6%, 0.3% and 0.2% respectively. Because the kefir and yogurt extracts were acidic (pH around 4.5). Dulbecco's Phosphate Buffered Saline (PBS) buffer was added to the culture media to adjust the pH between 7.0-7.6. Cells were incubated at 37° C. in a humidified atmosphere with 5% CO₂ for 8 days and the cell numbers in each well were counted using a Coulter Counter (Coulter Counter Corporation, USA). Each sample was run in quadruplicate. Control cells were incubated with the culture medium with the dosing vehicle (PBS).

Lactic Acid Concentration in Cell Cultural Media, Kefir and Yogurt Extracts:

After cells were collected for counting, the culture media were centrifuged at 4000 rpm for 10 min at 4° C. The supernatant was isolated for lactic acid measurement using a lactic acid assay kit from Sigma Diagnostics Inc. Kefir, milk and yogurt extracts were diluted 10 to 20 times before the measurement of lactic acid.

Statistics:

The cell numbers expressed as percentage of control from different treatments and dose were compared using two-way ANOVA. When the interaction between treatments and dose was also significant the difference between treatment groups was determined by Tukey's HSD multiple comparison test. All statistics test were performed using SAS 6.11 for PC (SAS, Cary, N.C.).

Results

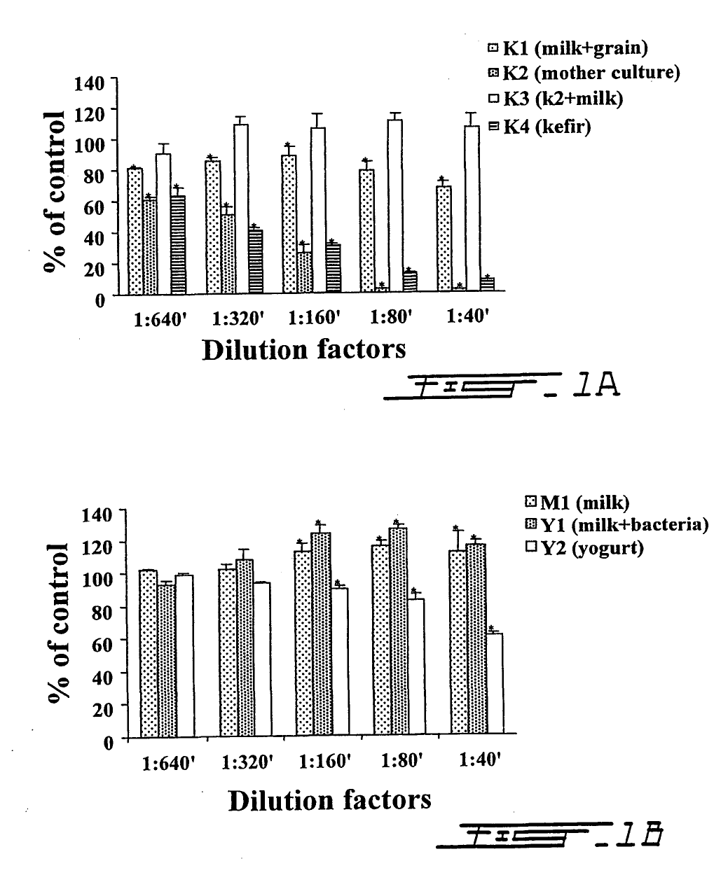

FIG. 1A showed effects of different kefir samples (K1-K4) on proliferation of MCF-7 cells. Values are means±SD (n=4) and * denotes significantly different from control at p<0.01. The effects of both the treatments and dose were significant (p<0.01). The mixture of kefir grain and milk (K1) showed a moderately inhibitory effect (p<0.01), whereas the fermented mother culture (K2) and the final kefir product (K4) showed a significantly stronger inhibitory effects effect p<0.01) in comparison to the K1 mixture. Dilution of the mother culture with milk (K3), however, resulted in elimination of inhibitory effects. In addition, the mother culture (K2) had significantly (p<0.05) more potent inhibitory effects on MCF-7 all proliferation than the kefir product (K4) at the 1:80 and 1:40 dilutions.

Yogurt (Y2) also showed similar inhibitory effects on the growth of the MCF-7 cells (FIG. 1B) but the inhibitory effect was significantly less than exhibited by the kefir extracts (p<0.01). Yogurt extract (Y2) at a 1:160 dilution in medium decreased the cell numbers by only 15% whereas kefir extract (K2) at 1:640 dilution in medium decreased the cell numbers by 40%. Milk (M1) and the mixture of milk and yogurt bacteria (Y1) both showed a slight but significant stimulation of cell growth beginning at 1:160 dilution in the media (FIG. 1B).

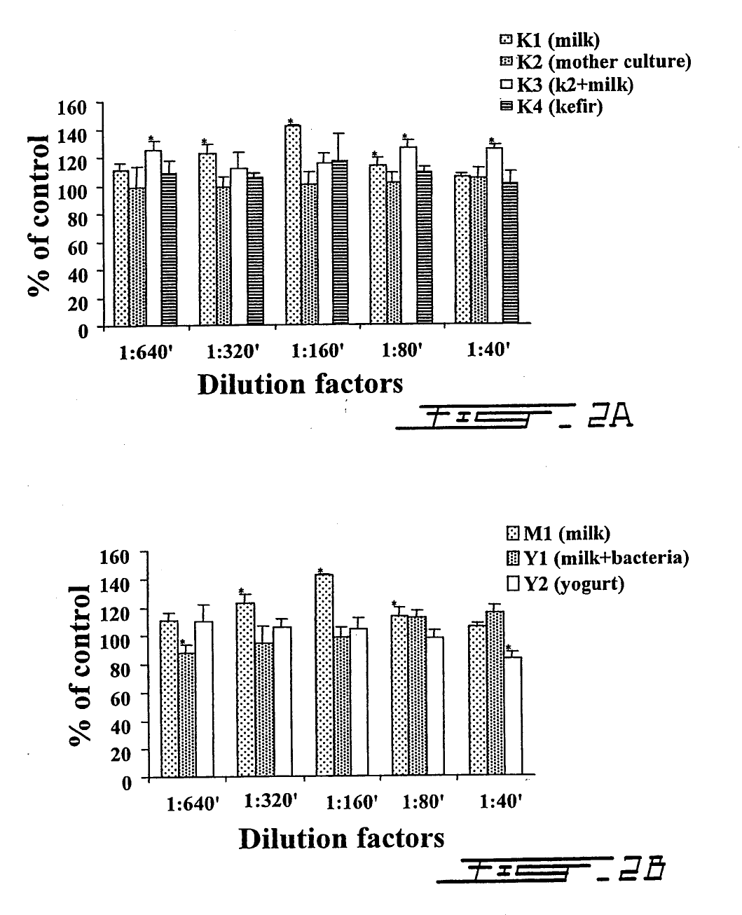

In contrast, both K2 and K4 kefir extracts did not effect the proliferation of the HMEC cells (FIG. 2A). Values are means±SD (n=4) and * denotes significantly different from control at p<0.01. The Y2 yogurt fraction, on the other hand, had a slight inhibitory effect on the growth of the HMEC cells (FIG. 2B) (p<0.05) and lowered the cell number to 83% at a 1:40 dilution in the medium. All the non-fermented milk products (K1, K3, M1 and Y1) showed a slight but significant (p<0.05) stimulation of cell growth (FIGS. 2A & 2B).

Discussion

Previous results have shown that yogurt exert anti-proliferative properties in MCF-7 cells (Biffi A et al., Nutrition and Cancer 28: 93-99, 1997). The present results show, however, that the anti-proliferative potency of kefir extracts on MCF-7 cellular growth is markedly greater than that of yogurt extracts and that, unlike yogurt, the kefir extracts do not suppress the growth of normal human mammary epithelial cells. Thus, this work is the first to indicate that a kefir fraction, unlike other fermented milk products, exerts anti-proliferative effects that are specific to tumor cells. The dose-response concentrations of the extracts used dilutions that varied from 1:640 to 1:40. The antiproliferative activity is clearly not caused by the yeast or bacteria of the kefir or yogurt as the test samples were filter sterilized. Likewise, lactic acid was excluded as the active ingredient since lactic acid measurements showed no relationship with lactic acid concentrations in the test mediums. Thus, the bioactive ingredient(s) must be a fermentation product other than lactic acid. As antiproliferative activity from the kefir extracts is observed in the MCF-7 cells but not the normal human mammary epithelial cells suggest that the active ingredients can bind to or triggers response that are specifically found in tumor cells. Previous work has shown that the administration of a polysaccharide isolated from the kefir grain had anti-tumor activities in mice (Shiomi M et al., Jap J Med Sci Bio. 35:75-80, 1982); however, the polysaccharides show no inhibitory effects against the growth and viability of cultured tumor cells and thus the anti-tumor effects are considered to be host mediated. Thus, the anti-proliferative agent in the kefir extract is unlikely to be a polysaccharide.

Milk proteins and peptides, especially those associated with whey, may be likely candidates as the bioactive ingredients of the kefir extracts. A number of whey proteins have been shown to have anti-carcinogenic properties and incubation of whey protein concentrates have been shown to increase proliferation of normal rat lymphocytes whereas the growth of rat mammary tumor cells was shown to be inhibited (Bourtourault M et al., CR Soc Biol 185, 319-323, 1991).

The present invention will be more readily understood by referring to the following examples which are given to illustrate the invention rather than to limit its scope.

### EXAMPLE I

Anti-Cancer Composition

In accordance with one embodiment of the present invention, there is provided a composition having anti-proliferative and/or inhibitory effects specifically targeted at malignant cells which comprises a filtrated bacteria-free and/or yeast-free liquid extract of initial fermentative kefir in association with a pharmaceutically acceptable carrier. This pharmaceutical composition will be administered in a physiologically acceptable medium for oral administration, e.g. deionized water, phosphate buffered saline (PBS), saline, plasma, proteinaceous solutions, aqueous glucose, alcohol, vegetable oil, or the like.

The composition may be lyophilized for convenient storage and transport.

The composition may also be administered parenterally, such as intravascularly (IV), intraarterially (IA), intramuscularly (IM), subcutaneously (SC), or the like. Administration may in appropriate situations be by transfusion. In some instances, administration may be nasal, rectal, transdermal or aerosol.

### EXAMPLE II

Prophylactic Composition

In accordance with one embodiment of the present invention, there is provided a prophylactic composition having neutraceutical properties, which comprises a filtrated bacteria-free and/or yeast-free liquid extract of initial fermentative kefir in association with a pharmaceutically acceptable carrier.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An anti-cancer composition having anti-proliferative and/or inhibitory effects specifically targeted at malignant cells, which comprises a filtrated bacteria-free and/or yeast-free liquid extract of initial fermentative kefir in association with a pharmaceutically acceptable carrier.

2. The anti-cancer composition of claim 1, wherein said filtrated extract is ultrafiltrated or microfiltrated.

3. The anti-cancer composition of claim 1 or 2, wherein said liquid extract comprises a protein concentration of 300 ng/ml to 5000 ng/ml.

4. The anti-cancer composition of claim 3, wherein said liquid extract comprises a protein concentration of about 313 ng/ml.

5. A method of inhibiting proliferation of malignant cells in patient, which comprises administering an effective amount of a filtrated bacteria-free and/or yeast-free liquid extract of initial fermentative kefir.

6. The method of claim 5, wherein said filtrated extract is ultrafiltrated or microfiltrated.

7. The method of claim 5 or 6, wherein said liquid extract comprises a protein concentration of 300 ng/ml to 5000 ng/ml.

8. The method of claim 5, 6 or 7, wherein said liquid extract comprises a protein concentration of about 313 ng/ml.

9. The method of claim 5, wherein said effective amount of a filtrated bacteria-free and/or yeast free liquid extract of initial fermentative kefir is administered orally.

10. The method of claim 5, wherein said malignant cells are selected from the group consisting of estrogen responsive cancer, cancer induced by oncovirus, hepatic cancer, colon cancer, prostate cancer, skin cancer and lung cancer.

11. The method of claim 10, wherein said estrogen responsive cancer is breast or uterine cancer.

12. Use of filtrated bacteria-free and/or yeast free liquid extract of initial fermentative kefir for inhibiting proliferation of malignant cells in a patient.

13. The use as claimed in claim 12, wherein said filtrated extract is ultrafiltrated or microfiltrated.

14. The use as claimed in claim 12 or 13, wherein said liquid extract comprises a protein concentration of 300 ng/ml to 5000 ng/ml.

15. The use as claimed in claim 12, 13 or 14, wherein said liquid extract comprises a protein concentration of about 313 ng/ml.

16. The use as claimed in claim 12, wherein said malignant cells are selected from the group consisting of estrogen responsive cancer, cancer induced by oncovirus, hepatic cancer, colon cancer, prostate cancer, skin cancer and lung cancer.

17. The use as claimed in claim 16, wherein said estrogen responsive cancer is breast or uterine cancer.

18. A prophylactic composition having neutraceutical properties, which comprises a filtrated bacteria-free and/or yeast-free liquid extract of initial fermentative kefir in association with a pharmaceutically acceptable carrier.

19. The prophylactic composition of claim 18, wherein said filtrated extract is ultrafiltrated or microfiltrated.

20. The prophylactic composition of claim 19, wherein said liquid extract comprises a protein concentration of 300 ng/ml to 5000 ng/ml.

21. The prophylactic composition of claim 20, wherein said liquid extract comprises a protein concentration of about 313 ng/ml.
